(19) United States
(12) Patent Application Publication
(10) Pub. No.: US 2018/0273895 A1
(43) Pub. Date: Sep. 27, 2018

(54) LACTIC ACID BACTERIA AND THEIR USE FOR THE TREATMENT OF MASTITIS

(71) Applicant: BioGaia AB, Stockholm (SE)

(72) Inventors: Bo Möllstam, Lerum (SE); Eamonn Connolly, Lidingö (SE)

(21) Appl. No.: 15/543,974

(22) PCT Filed: Jan. 14, 2016
(86) PCT No.: PCT/EP2016/050699

§ 371 Date: Jul. 14, 2017

Related U.S. Application Data
(60) Provisional application No. 62/104,164, filed on Jan. 16, 2015.

(51) Int. Cl. C12N 1/20; C12R 1/225; A61K 35/747; A61K 9/00; C12Q 1/6883

(52) U.S. Cl. CPC C12N 1/20; C12R 1/225; C12Q 1/6883; A61K 9/0014; A61K 35/747

(57) ABSTRACT

The present invention relates to novel Lactic Acid Bacteria strains Lactobacillus reuteri selected from the group comprising Lactobacillus reuteri DSM 32229, Lactobacillus reuteri DSM 32230, Lactobacillus reuteri DSM 32231 and Lactobacillus reuteri DSM 32232 and products comprising these strains. The present invention also relates to a use of one or more Lactic Acid Bacteria strains as a probiotic for the treatment of an inflammation and infection, such as mastitis and/or thrush, especially after topical administration of said strains.

This application is a national phase entry pursuant to 35 U.S.C. § 371 of International Application No. PCT/EP2016/050699, filed Jan. 14, 2016, which claims the benefit of U.S. Provisional Application No. 62/104,164, filed Jan. 16, 2015, each of which is incorporated by reference herein in its entirety for any purpose.

This application contains a sequence listing submitted in electronic format. The file name is “2017-10-05_01189-0003-00US_Seq_List_ST25,” it was created on Oct. 5, 2017, and is 659 bytes in size.

### FIELD OF THE INVENTION

The present invention relates to novel Lactic Acid Bacteria strains and products comprising these strains. The present invention also relates to a use of one or more Lactic Acid Bacteria strains as a probiotic for the treatment of inflammation and infection, such as mastitis and/or thrush.

### BACKGROUND OF THE INVENTION

Certain strains of lactic acid producing bacteria, such as Lactobacillus and Bifidobacterium are commonly used as probiotics in various types of products, such as foods, food-supplements and cosmetic or pharmaceutical compositions. The word “probiotic” stems from the Greek word pro, which means “promoting” and biotic, which means “life”. The Food and Agricultural Organization of the United Nations define probiotics as “live microorganisms which, when administered in adequate amounts, confer a health benefit on the host”. Growth and colonization of harmful microorganisms can be prevented by lactic acid producing bacteria through their competitive colonization on or inside the mammal, or through formation of biofilms, or through competition of available nutrients or other mechanisms. Bacteria may be useful through their production of specific substances such as hydrogen peroxides, bacteriocins, organic acids (including lactic acid and acetic acid) that lower the pH in a fluid, such that growth of harmful bacteria can be prevented. A mammal can benefit from probiotic bacteria through many ways. The effectiveness of the probiotic bacteria is mostly strain-specific, where each strain or groups of strains may contribute to the health of the mammal through different specific mechanisms. Probiotics can for example prevent or inhibit the proliferation of pathogens, suppress production of virulence factors by pathogens, or modulate the immune response in a pro-inflammatory or an anti-inflammatory way.

Mastitis is an infection of the breast tissue that results in breast pain, swelling, warmth and redness of the breast or udder. Mastitis is an infection that affects all mammalian species and is mainly caused by a bacterial infection (infectious mastitis) and then in most cases by Staphylococcus aureus. Mastitis is different from a blocked duct, because a blocked duct is not thought to be an infection and thus does not need to be treated with antibiotics. Blocked milk ducts are sometimes referred to as non-infective mastitis. The difference between a “mild” mastitis and a “severe” blocked duct may not be easy to determine and it is possible that a blocked duct eventually evolves into mastitis.

Infectious mastitis is of utmost importance to prevent in animals that are used for milk production, such as cows, camels, ewes or goats, since milk produced during the infection and treatment must be discarded. Mastitis most commonly affects mammals, including women, who are breast-feeding (lactation mastitis), although mastitis can occur in mammals who are not breast-feeding.

Some antibiotics that are targeted to Staphylococcus aureus include: cephalexin, cloxacillin, dicloxacillin, flucloxacillin, amoxicillin combined with clavulinic acid, clindamycin and ciprofloxacin. Other examples may be methicillin-resistant Staphylococcus aureus (CA-MRSA), such as cotrimoxazole and tetracycline. However, in many cases it is better to avoid antibiotics, because antibiotics may make other infections possible and are also known to cause dysbiosis and a shift in the microbiota in the mammal. Besides, a mammal body may heal from mastitis by other means without interference from antibiotics, thereby avoiding potential antibiotic resistance issues.

Probiotics have previously been demonstrated as an alternative approach to the use of antibiotics, to treat and prevent mastitis in humans. Certain types of Bifidobacterium and Lactobacillus has been tested (Sytnik, S. I. et al. (Vrach Delo., 1990, 3:98-100), Greene, W. A. et al. (J. Dairy Sci., 1991, 74:2976-2981)). For example, U.S. Pat. No. 4,591,499 describes methods to treat mastitis by an intramammary injection of an oil-in-water emulsion containing lactobacillus strains that are non-pathogenic. In the prior art, the probiotic is administered orally or parenterally, e.g. subcutaneous or intermuscular.

WO2008145756 discloses a process for the selection of the probiotic bacterial strains by (i) isolating lactic acid bacteria or bifidobacteria strains present in fresh milk from a mammalian species by selection in lactic acid culture media, (ii) selecting those strains from step (i) that are capable of being transferred to the mammary gland after oral intake and/or colonize the mammary gland after topical application, (iii) selecting those strains from step (ii) which are able to reduce the rates of survival and/or the rates of adhesion to epithelial cells of Staphylococcus aureus, by the production of cytokines, and (iv) selecting those strains from step (iii) that are capable of protecting animals from mastitis. In step (ii), the strains isolated in step (i) are selected based on their ability to being transferred to the mammary gland after oral intake and/or colonize the mammary gland. For detecting the ability to being transferred to the mammary gland, an assay such as described in WO2004003235 for detecting transfer of a microorganism to the milk after oral intake can be used.

US20030235560 discloses lysogenic bacteriophages that are used specifically towards bacteria that cause mastitis in milk-producing cattle, and a composition used for application on mammal's udders.

US20070077235 discloses methods and compositions for treating bacterial infections, such as mastitis in milk-producing cattle, using bacteria-associated phage proteins, enzymes or peptides, and/or peptide fragments thereof. More specifically, US 20070077235 discloses phage lytic and/or holin proteins, or peptides and peptide fragments thereof, blended with a carrier for the treatment and prophylaxis of bacterial infections like mastitis.

Histamine is an organic nitrogen compound involved in several health-associated processes of a mammal, including local immune responses as well as regulating physiological function in the gut and acting as a neurotransmitter. As part of an immune response to foreign pathogens, histamine may be produced by basophils and mast cells. Histamine can be derived from the decarboxylation of the amino acid histidine, a reaction catalyzed by the enzyme L-histidine decarboxylase. Such production of histamine by certain bacterial strains has up until recently been seen as a health risk rather than a possible benefit for humans. For example, Sconnbroid poisoning is due to histamine production by bacteria in spoiled food, particularly fish. Contrary to this type of histamine production, local production of controlled amounts of histamine from selected bacteria can give beneficial effects rather than detrimental effects. Certain bacteria are capable of producing anti-inflammatory histamine using histidine decarboxylase enzymes unrelated to those found in eukaryotes, as described in US2013149291.

A selected group of Lactic Acid Bacteria strains, including certain strains of Lactobacillus reuteri, can produce histamine and such histamine, contrary to earlier belief, may benefit the mammal by reduction of inflammation. Lactobacillus reuteri is considered an indigenous organism of the human gastrointestinal tract and is present for example on the mucosa of the gastric corpus, gastric antrum, duodenum, and ileum.

WO2013/011137 discloses novel Lactobacillus strains useful for the treatment of inflammation disorders in various locations of the body after oral administration of the Lactobacillus strain, alternatively together with an additional histidine source. Said Lactobacillus convert histidine into histamine inside the body. Histamine is used to treat the inflammatory disorder.

There are large losses in profit to dairy producers due to mastitis, including reduced milk production, discarded milk, uses of antibiotic drugs, veterinary services and labor. This motivates research to improve methods of treatment. Further, the use of antibiotics for the treatment of mastitis can lead to unwanted side effects and may increase the antibiotic resistance. The present invention aims to solve health-related issues associated with the condition of mastitis.

### OBJECT AND SUMMARY OF THE INVENTION

It is an object of the present invention to at least partly overcome the above problems, and to provide new Lactic Acid Bacteria strains (Bacteria).

This object is achieved by selecting and using Bacteria strains that have the ability to convert histidine into histamine, wherein the histidine is available to the Bacteria by means of a histidine comprising body fluid inside or outside of a body. Such body fluid can be milk. The histamine provides an anti-inflammatory effect. The Bacteria are selected by the presence of genes needed to convert histidine to histamine, using for example polymerase chain reaction (PCR). The Bacteria have the ability to convert histidine into histamine in a fluid having a concentration of histidine of more than 5, or 10 mg/100 ml, or between 1 and 75 mg/100 ml fluid, or between 10 and 50 mg/100 ml, or between 10 and 40 mg/100 ml fluid or breast milk. The Bacteria can be used without advert effects that may be caused by histamine. The Bacteria may be administered orally, parenterally or topically. In one embodiment, the Bacteria is administered orally. In another embodiment, the Bacteria is topically administered and histidine is available on the skin or mucosa of the body.

The object of the invention is achieved by a biologically pure culture of a Lactobacillus reuteri strain selected from the group comprising or consisting of Lactobacillus reuteri DSM 32229, Lactobacillus reuteri DSM 32230, Lactobacillus reuteri DSM 32231 and Lactobacillus reuteri DSM 32232.

In yet a further embodiment, the one or more Lactic Acid Bacteria strain is selected from the group comprising or consisting of Lactobacillus reuteri ATCC PTA 6475, Lactobacillus reuteri ATCC PTA 4659, Lactobacillus reuteri ATCC PTA 5289 and Lactobacillus reuteri ATCC PTA 5290. The histidine operon comprises three genes (the histidine/histamine antiporter, the histidine decarboxylase pyruvoyl type A (HdcA), and histidine decarboxylase pyruvoyl type B (HdcB). These Bacteria can produce histamine from histidine. Bacteria not having the histidine operon cannot produce histamine from histidine.

Another object of the invention relates to a use of one or more Lactic Acid Bacteria strains for treatment of mastitis, whereby said Bacteria has an ability to convert histidine, present in a topical body fluid, into histamine. The use may be medical or cosmetic. No additional external histidine is needed for the treatment.

Thrush is a fungal infection caused by accumulation of Candida strains. It can affect anyone but it is more likely to affect the oral cavity of a baby. A baby with thrush is believed to increase the risk for the lactating mother to develop mastitis.

The selected Lactic Acid Bacteria strains of the invention as defined herein also have the advantage of producing and secreting antimicrobial compounds, such as lactic acid. Candida strains are susceptible to such antimicrobial compounds, including the lowering of local pH by lactic acid. The use of the Lactic Acid Bacteria strains, especially those mentioned above, may therefore also be beneficial for the prevention or treatment of thrush. Said Bacteria strains may also be used for the prevention or treatment of mastitis in a mammal whose newborn or baby is infected with thrush.

In an embodiment, one or more Lactic Acid Bacteria strain is used for a treatment of mastitis and/or thrush. In one embodiment, the treatment is a topical treatment.

In another embodiment, the body fluid is milk present outside the body of a mammal to be treated.

Said one or more Lactic Acid Bacteria strain have the ability to, from the outside of the body, convert the natural amounts of histidine found in the treated mammal's own milk, produced from the mammary gland, into histamine and thus make it possible for a topical product comprising said Bacteria, to provide local anti-inflammatory effects to the mammal from the outside of the body. This allows treatment of mastitis by applying the Bacteria on the skin or mucosa of the body without addition of external histidine. The Bacteria have the ability to convert histidine into histamine in a fluid having a concentration of histidine of more than 5, or 10 mg/100 ml, or between 1 and 75 mg/100 ml fluid, or between 10 and 50 mg/100 ml or between 10 and 40 mg/100 ml fluid or breast milk. Topical administration is less invasive for a patient and is believed to improve treatment compliance by the patient.

In a further embodiment, the one or more Lactic Acid Bacteria strain is selected based on their ability to convert natural amounts of histidine, present in milk, into histamine, outside the body of a mammal.

In another embodiment a Lactobacillus reuteri strain with the ability to convert natural amounts of histidine, present in milk, into histamine, outside the body of a mammal, is selected.

In another embodiment, the one or more Lactic Acid Bacteria strain is selected from the group comprising or consisting of Lactobacillus reuteri DSM 32229, Lactobacillus reuteri DSM 32230, Lactobacillus reuteri DSM 32231 and Lactobacillus reuteri DSM 32232 for use for a treatment of mastitis and/or thrush. In yet a further embodiment, the one or more Lactic Acid Bacteria strain is selected from the group comprising or consisting of Lactobacillus reuteri ATCC PTA 6475, Lactobacillus reuteri ATCC PTA 4659, Lactobacillus reuteri ATCC PTA 5289 and Lactobacillus reuteri ATCC PTA 5290 for use for a treatment of mastitis and/or thrush.

The advantage of said Lactic Acid Bacteria or a composition comprising said Bacteria, is their ability to convert histidine into histamine upon contact with a histidine comprising fluid. According to the invention herein said fluid can be outside of the body. The location of the histidine is not important for the Lactic Acid Bacteria mentioned above. This means that these Lactic Acid Bacteria can be applied topically to the body of a mammal. Once in contact with the histidine comprising fluid, the Lactic Acid Bacteria may start to migrate into the body through one or more gland openings. The Lactic Acid Bacteria thus provides for a non-invasive method of treating mastitis and/or thrush.

The invention also relates to a method of preventing and/or treating mastitis and/or thrush comprising administering to a mammal, such as a human, in need thereof, a therapeutically effective amount of the one or more Lactic Acid Bacteria, as defined above.

The invention also relates to a use of one or more Lactic Acid Bacteria strain, as defined above, in relieving an inflammation disorder connected to a condition of blocked gland ducts. The invention further relates to a use of a one or more Lactic Acid Bacteria strain, as defined above, in alleviating and/or preventing recurring an inflammation disorder of a milk system (e.g. breast or udder) of a mammal.

The invention also relates to a use of one or more Lactic Acid Bacteria strain, as defined above, in the manufacture of a pharmaceutical product, medical device, such as a cream, ointment or an oil, or a cosmetic product, for the treatment and/or prevention of a disease, disorder or condition, such as inflammation, mastitis and/or thrush.

When activated by the contact with milk from the mammary gland, the duct or the nipple, the one or more Lactic Acid Bacteria strain may colonize or in other ways be in contact with the milk system of the mammal and provide beneficial effects, including anti-inflammatory and anti-bacterial effects, to the mammal. The selected Lactic Acid Bacteria strains provide local production of histamine and thus prevent and/or reduce bacterial infections, without the need for additional histidine, i.e. without adding external histidine to the administered Bacteria. Female mammals who suffer from a condition of mastitis during lactating, would thus benefit from effects that could reduce inflammation and reduce the bacterial infection that causes mastitis and/or thrush.

The mammal may be a human, a cow, a dog, a cat, a camel, a ewe and a goat, or any other milk producing mammal.

The object of the invention is also achieved by a method for topical administration of one or more Lactobacillus reuteri strain, whereby the strain is applied to a skin or mucosa of the mammal using an adsorbent or non-adsorbent product. In one embodiment, the Lactobacillus reuteri is selected from the strains defined above.

In another embodiment of the method, the adsorbent or non-adsorbent product is a pad, a wipe and/or a tissue made of adsorbent or non-adsorbent material, whereby the material is suitable as a carrier for the Lactobacillus reuteri strain. In one embodiment, the product is adapted to be placed in contact with the mammary gland of the mammal by wiping.

In a further embodiment, the adsorbent or non-adsorbent product is contained or surrounded by cotton wadding, wool or a wool-like material with characteristics that are similar to wool.

In a further embodiment of the method, the cotton wadding, wool or a wool-like material is adapted to provide heat. Such heating effects may help reduce the inflammation of the mammary gland, help draining the mammary gland, reduce pain, increase comfort and furthermore provide optimal growth conditions for the probiotic Bacteria that are administered topically to the mammary gland for colonization.

The one or more Lactic Acid Bacteria strain, as defined above, may be applied locally at the site or in the proximity of the site of the inflammation disorder. This provides for a more effective and efficient manner of treatment compared to the prior art methods. Topical application combined with close proximity to the site of the inflammation disorder allows for a reduction in amount of Lactic Acid Bacteria needed to treat the disorder. This in turn reduces costs for treatment.

The invention further relates to a composition comprising the one or more Lactic Acid Bacteria strain, as defined above, in the association with an acceptable carrier.

The invention also relates to a process for the preparation of a pharmaceutical or cosmetic composition, as defined above, which comprises mixing cultures of the one or more Lactic Acid Bacteria strain, as defined above, with an acceptable carrier. A carrier may for example be a lipid or additive.

Another object of the invention relates to a composition comprising cultures of the one or more Lactic Acid Bacteria strain, as defined above, in a dried form optionally together with an anti-moisture agent and one or more additive. In one embodiment, the cultures of the one or more Lactic Acid Bacteria strain are in a lyophilized form.

Normally, preserved Lactic Acid Bacteria demonstrate rapid loss of viability in moist or even in semi-moist conditions, and it is therefore of great importance that the Bacteria are not exposed to moisture during storage. The problem can in part be handled by supplying a product comprising said Bacteria and drying said product to remove the moisture, and/or by adding one or more anti-moisture agents to said product. The cultures of the one or more Lactic Acid Bacteria strain may be preserved in a freeze-dried or lyophilized form.

In another embodiment, the anti-moisture agent is a lipid selected from the group of plant derived lipids or a polymer. The polymer is selected from the group comprising polyvinyl alcohol, polyethylene oxide, polyvinyl pyrrolidone and starch. In one embodiment, the lipid is selected from the group comprising olive oil, canola oil, coconut oil, palm kernel oil, peanut oil, soybean oil, dimethicone, paraffin oil and petrolatum.

By suspending the cultures of one or more Lactic Acid Bacteria strain in a form where the Bacteria are in an environment with low water activity, such as dried, preferably freeze-dried form, together with one or more lipids, the bacterial survival is increased. Lipids enhance transfer rates of Bacteria to the skin area and the lipids also increases the survival of Bacteria when delivered to the skin by creating a suitable microenvironment. Survival of the cultures of the one or more Lactic Acid Bacteria strain and transfer rates of the Bacteria to the skin area are expected to be improved by the use of these lipids. Lipids may have a further advantage of interacting with skin lipids. This will smoothen the skin. Especially at the site of inflammation, the lipid may provide a synergistic effect of healing the skin.

In a further embodiment, the composition comprises one or more additives selected from the group comprising carbohydrates, C₆₋₁₂ medium chain fatty acids, emollients, surfactants, emulsifiers, proteins, amino acids, polyols, silica and antioxidants. These additives are readily available at relatively low cost.

In yet another embodiment, the composition comprises

| | |
|---|---|
| a) C₆₋₁₂ medium-chain triglycerides (MCT) | 25-48.5 wt % |
| b) sunflower oil | 25-48.5 wt % |
| c) silica dioxide | 0-1 wt % |
| d) one or more Lactic Acid Bacteria (dried) | 0.5-2 wt % |

whereby weight percentages (wt %) are percentages of the total weight of the composition, and the activity of the Lactic Acid Bacteria is between 10⁵-10¹² CFU (colony forming units) per gram.

In one embodiment, the activity of the Lactic Acid Bacteria is between 10⁷-10⁸ CFU per gram.

It is believed that this composition can be used for the treatment of mastitis. See the experimental data outlined below.

In an embodiment of the composition, the one or more Lactic Acid Bacteria strain is selected from the group comprising or consisting of Lactobacillus reuteri DSM 32229, Lactobacillus reuteri DSM 32230, Lactobacillus reuteri DSM 32231 and Lactobacillus reuteri DSM 32232. In another embodiment of the composition, the one or more Lactic Acid Bacteria strain is selected from the group comprising or consisting of Lactobacillus reuteri ATCC PTA 6475, Lactobacillus reuteri ATCC PTA 4659, Lactobacillus reuteri ATCC PTA 5289 and Lactobacillus reuteri ATCC PTA 5290.

A further aspect of the invention relates to a use of the composition for the treatment of mastitis and/or thrush. In one embodiment, the treatment is a topical treatment.

Advantages of the composition, as well as the preferred embodiments thereof, are apparent from the discussion above with reference to the Lactic Acid Bacteria and uses thereof.

The invention further relates to a method for selection of Lactic Acid Bacteria strains, whereby said Bacteria has an ability to convert histidine, present in a topical body fluid, into histamine as described in Example 1.

In one embodiment, the method for selection of Lactic Acid Bacteria strains, comprises the steps of screening and selection for strains of Lactic Acid Bacteria, which have an active histidine operon using a PCR method as further described in Example 5.

### BRIEF DESCRIPTION OF THE DRAWINGS

The following drawings are provided to illustrate various aspects of the present inventive concept and are not intended to limit the scope of the present invention unless specified herein.

FIG. 1 represent a schematic illustration of the mammary gland and different conditions described; a. the mammary gland under normal conditions; b. the mammary gland under inflammation (blocked duct or similar condition); c. the mammary gland during infection and mastitis. Grey filled circles illustrates inflammation, ellipses with checkered filling illustrate bacteria during infection, and the arrow illustrate a milk flow.

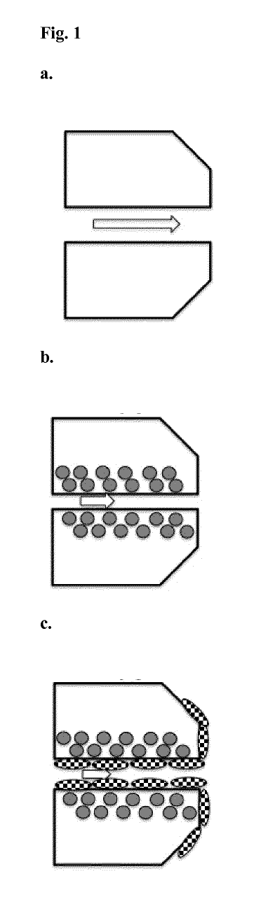

### DETAILED DESCRIPTION OF PREFERRED EMBODIMENTS OF THE INVENTION

### Definitions

The term “treatment” as used herein is understood to include prevention, reduction and prophylaxis.

The term “Bacteria” as used herein is understood to include Lactic Acid Bacteria strains including, but not limited to, any specific Lactic Acid Bacteria strain mentioned herein.

The term “topical” as used herein is understood to refer to an application or administration to a particular place on or in the body, as opposed to systemically.

The term “disorder” as used herein is understood to include disease and condition.

The term “non-invasive” is understood to be a treatment done without cutting the body or putting something into the body, e.g. the term is understood to be a topical administration on the skin or mucosa of the mammal.

### Lactic Acid Bacteria

Lactobacillus reuteri strain 93a has been deposited under the Budapest Treaty at DSMZ (Leibniz Institute DSMZ—German Collection of Microorganisms and Cell Cultures, Inhoffenstr. 7B, D-38124 Braunschweig, Germany) on Dec. 11, 2015, and has been given the accession number DSM 32229.

Lactobacillus reuteri strain F33 has been deposited under the Budapest Treaty at DSMZ (Leibniz Institute DSMZ—German Collection of Microorganisms and Cell Cultures, Inhoffenstr. 7B, D-38124 Braunschweig, Germany) on Dec. 11, 2015, and has been given the accession number DSM 32232.

Lactobacillus reuteri strain C30 has been deposited under the Budapest Treaty at DSMZ (Leibniz Institute DSMZ—German Collection of Microorganisms and Cell Cultures, Inhoffenstr. 7B, D-38124 Braunschweig, Germany) on Dec. 11, 2015, and has been given the accession number DSM 32230.

Lactobacillus reuteri strain D276 has been deposited under the Budapest Treaty at DSMZ (Leibniz Institute DSMZ—German Collection of Microorganisms and Cell Cultures, Inhoffenstr. 7B, D-38124 Braunschweig, Germany) on Dec. 11, 2015, and has been given the accession number DSM 32231.

Lactobacillus reuteri ATCC PTA 6475, Lactobacillus reuteri ATCC PTA 4659, Lactobacillus reuteri ATCC PTA 5289 and Lactobacillus reuteri ATCC PTA 5290 are Lactobacillus reuteri strains owned by Biogaia AB, Sweden.

The strains of Lactic Acid Bacteria may be screened for and selected on the basis of the presence of genes needed to convert histidine into histamine using PCR, or other relevant method, as described in Thomas C M et al. (2012) PLoS ONE 7(2): e31951. doi:10.1371/journal.pone. 0031951, which is hereby incorporated herein by reference in its entirety.

A biologically pure culture of these Lactobacillus reuteri can be obtained by a selection method comprising the steps of;
screening the Lactic Acid Bacteria strains for the presence of an active histidine operon using a PCR method with two primers according to the following: sequence (CGTCAYTATCCWGCTCCWGG) (SEQ ID NO: 1) referred to as hdcA425fde and sequence (TCCATRTCAGTATCWGGKGT) (SEQ ID NO: 2) referred to as hdcA867rde. The primers were designed from an alignment of hdc from L. reuteri, L. hilgardii, L. buchneri and L. sakei. The PCR analysis is further described in Example 5 and;
selecting a strain, which has an active histidine operon.

The Lactic Acid Bacteria can also be selected based on their ability to convert histidine, present in a body fluid, into histamine, as described in Example 1. In one embodiment, the method comprises the steps of collecting samples of breast milk, determining the amount of histidine in the milk, incubating the samples with the Bacteria to be tested anaerobically at about 37° C. or other suitable temperature for 20 to 30 hours, determining the amount of histamine in the samples and selecting Bacteria strains that has an ability to convert histidine to histamine.

### Medical and Cosmetic Use

L. reuteri DSM 32229, L. reuteri DSM 32230, L. reuteri DSM 32231 and L. reuteri DSM 32232 are believed to be useful for the treatment of mastitis and/or thrush, especially after topical administration to a mammal.

Examples of other Lactic Acid Bacteria strains that have an ability to convert histidine present in a body fluid into histamine, may be Bacteria selected from the group comprising L. reuteri ATCC PTA 6475, L. reuteri ATCC PTA 4659, L. reuteri ATCC PTA 5289 and L. reuteri ATCC PTA 5290.

Lactic Acid Bacteria strains, especially Lactobacillus reuteri strains, more specifically the Lactic Acid Bacteria strains mentioned above, are believed to be useful for the treatment of mastitis and/or thrush, especially after topical administration to a mammal. The Bacteria, as defined above, are useful for treatment as mentioned above, without addition of external histidine sources, e.g. said Bacteria use only histidine from the milk of the mammal to be treated.

The Bacteria strains, as defined above, are also believed to be useful in relieving an inflammation disorder connected to a condition of blocked gland ducts and in alleviating and preventing recurring an inflammation disorder of a milk system (e.g. breast or udder) of a mammal.

The Bacteria strains, as defined above, can be administered topically to provide means for local production of histamine at the proximity and inside the breast, breast nipple, udder and mammary gland of the mammal, by utilizing and converting the natural amount of histidine present in the milk produced by the mammary gland, to histamine. By administrating one or more Lactic Acid Bacteria strains, as defined above, topically at a site of the milk system or at an inflamed site of the mammal, and combine the administered one or more Lactic Acid Bacteria strain, as defined above, with the milk from the mammal, said Bacteria become activated and start to produce histamine by converting histidine present in the milk.

The local histamine has an anti-inflammatory effect and is used to treat the inflammation. At the same time, the Bacteria may use the milk to migrate from the outside of the body into the mammary gland of the mammal. Thus, Lactic Acid Bacteria strains, as defined above, provide an anti-inflammatory and/or anti-bacterial effect and/or anti-infectious effect to a mammal from the outside of the body and without the addition of external histidine, i.e. histidine not from the milk of the mammal to be treated.

The mammal may be a human, a cow, a dog, a cat, a camel, a ewe and a goat, or any other milk producing mammal.

The Bacteria strains, as defined above, may also be used for cosmetic treatment.

The natural amount of histidine present in breast milk has thoroughly been studied in several studies (EC Scientific Committee on Food (2003) Report on the Revision of Essential Requirements of Infant Formulae and Follow-on Formulae. European Commission Health and Consumer Protection Directorate—General, European Commission, Brussels), resulting in an average concentration of 29 mg/100 ml of breast milk, or 24 mg/g total crude protein. Furthermore, breast milk is known to provide all various types of nutrients for the Lactic Acid Bacteria to propagate and colonize the mammary gland.

### Composition

The composition may be a pharmaceutical composition or cosmetic composition or a device or the like.

In order to prevent rapid loss of viability in moist or even in semi-moist conditions, it is of great importance that the Bacteria are not exposed to moisture during storage. The problem can in part be handled by supplying product with said Bacteria and drying said products to remove the moisture and finally provide said product in moisture impervious packages.

In a composition comprising one or more Lactic Acid Bacteria strains, the Bacteria are preferably used in a dried form, such as a freeze dried form or a lyophilized form.

To protect the preserved Bacteria against moisture, the Bacteria may be dispersed in one or more hydrophobic substance or anti-moisture agent, which due to its hydrophobic character prevent moisture to reach the embedded bacterial cells. Optionally, one or more additive may be added to the composition.

Lipids may be used as anti-moisture agents. Examples of lipids include petroleum-derived lipids, synthetic lipids and animal- or plant-derived lipids. The one or more lipids may be selected from the group comprising olive oil, canola oil, coconut oil, palm kernel oil, peanut oil, soybean oil, dimethicone, paraffin oil, and petrolatum.

Polymers may also be used as anti-moisture agents. Polymers that are suitable to protect the bacterial cells from moisture during storage and transport, can preferably be dissolved in bodily fluids to release the bacterial cells when exposed to wet or moist conditions. The polymers should be non-toxic and non-irritant to a mammal skin. The one or more polymers may be selected from the group comprising polyvinyl alcohol, polyethyleneoxide, polyvinyl pyrrolidone and starch.

Additional components may be added to protect the Bacteria during the manufacturing of the products containing the Bacteria, e.g. carbohydrates, such as maltose, sucrose, trehalose, lactose, glucose and fructose; proteins, such as skim milk and albumin; amino acids, such as Na-glutamate; polyols, such as xylitol, mannitol and sorbitol; and antioxidants, such as Na-ascorbate. Some of the components described above may also be utilized by the Bacteria as nutrients for their propagation, once the Bacteria start to colonize on and in the mammal. Preferred growth conditions will contain a carbon source, in particular glucose, which will support the production of histamine by Lactobacillus reuteri strains (Thomas C M et al. (2012) PLoS ONE 7(2): e31951. doi:10.1371/journal.pone. 0031951). Preferably, the growth conditions are not dependent on sucrose as a source of carbon, or at least will only comprise sucrose at such a level that will not significantly compromise histamine production by the Lactobacillus reuteri strain.

The composition may comprise further additives selected from the group comprising carbohydrates, C₆₋₁₂ medium chain fatty acids (MCT), emollients, surfactants, emulsifiers and silica.

The composition or device may be selected from a lotion, cream, ointment, oil, salve, liniment, embrocation, rub, gel, petroleum jelly, balm, emollient, unguent, balsam, and the like.

An example of a composition according to the invention may be a composition comprising

| | |
|---|---|
| a) C₆₋₁₂ medium-chain triglycerides | 20-49.5 wt %, or 25-48.5 wt % |
| (MCT) | |
| b) sunflower oil | 20-49.5 wt %, or 25-48.5 wt % |
| c) silica dioxide | 0-5 wt %, or 0-1 wt % |
| d) one or more Lactic Acid Bacteria | 0.1-5 wt %, or 0.5-2 wt % |
| (dried) | |

and lipids, e.g. sunflower oil or MCT, up to 100%.

The weight percentages (wt %) are percentages of the total weight of the composition. The activity of the Lactic Acid Bacteria may be between 10⁵-10¹² CFU per gram or between 10⁷-10⁸ CFU per gram.

The concentration of the histamine producing Lactobacillus reuteri strains in the composition should be selected in such way that the desired effect is achieved without causing adverse effects. In some embodiments, the concentration by weight of certain histamine producing Lactobacillus reuteri strains ranges from about 0.01 to about 10 wt %, or from about 0.1 to about 10 wt %, or from about 0.1 to about 5 wt %, or from about 0.5 to about 5 wt % of the total weight of the composition. The activity of the used Bacteria may be between 10⁵-10¹² CFU per gram or between 10²-10⁸ CFU per gram of Bacteria culture.

These concentration levels may correspond to a one, two, three or four times daily topical application of the composition.

### Administration

The invention also provides a method for placing the probiotic Bacteria, as defined above, in contact with the milk system of the mammal. A topical composition comprising said Bacteria may be applied to the skin at a concentration of the histamine producing Bacteria strains that is sufficient to treat mastitis. For example at the concentrations mentions above.

The method may comprise applying the Bacteria, as defined above, or a composition comprising said Bacteria, to the skin or mucosa of the mammal using an adsorbent or non-adsorbent product, for example, a tissue, a pad or a wipe. The composition may be incorporated in an adsorbent product, such as a tissue, a pad or a wipe and sealed prior to use. For example, the composition may be soaked into a tissue, pad or wipe, vacuum dried and sealed. The product can be placed in contact with the mammary gland of the mammal by wiping.

The method may also comprise applying the Bacteria, as defined above, in a composition comprising said Bacteria, selected from a lotion, cream, ointment, oil, salve, liniment, embrocation, rub, gel, petroleum jelly, balm, emollient, unguent, balsam, and the like to the skin or mucosa of the mammal.

The composition may be applied on the nipple, the areola, and the whole breast, plus eventual areas of redness on the breast. The Lactic Acid Bacteria or a composition comprising said Bacteria, may be administered using pads made of adsorbent, or non-adsorbent material, which can be placed in contact with the milk system or mammary gland of the mammal, for example as an insert in an undergarment bra. Such pads are preferably compatible with and suitable as a carrier for viable, but dormant probiotic Bacteria, especially the Lactic Acid Bacteria as defined above. The pad is for example a nursing pad. The pads may be contained or surrounded by cotton wadding, wool or a wool-like material with characteristics that are similar to wool. The pads may be adapted to provide an insulation or heating effect to the skin of the mammal. Such heating effect may be provided by the body itself in combination with a covering and insulating garment.

The adsorbent or non-adsorbent product may decrease, eliminate and/or prevent the condition of mastitis and/or thrush. Such effects may be evaluated clinically, objectively and/or subjectively by a health care professional, a treatment subject or an observer. The treatment may be carried out for one week, two weeks, three weeks or as long as breast feeding takes place. Preferably, treatment is carried out three times per day for two weeks. Alternatively, the Bacteria, as defined above, or a composition comprising said Bacteria, may be provided in a kit comprising the Bacteria as defined above and an acceptable carrier system. The kit may include other items useful in the handling, preparation and use of the composition as well as instructions for use of the same.

### EXAMPLES

The present invention will now be described with reference to the following examples. These examples are for the purpose of illustrating aspects of the present invention, and are not intended to limit the scope of the invention as defined by the claims.

### Example 1

Three test products are made consisting of:
1. Freshly grown Lactobacillus reuteri strain DSM 17938 in LDM
2. Freshly grown Lactobacillus reuteri strain ATCC PTA 4659 in LDM
3. Lactobacillus Defined Media (LDM) alone
   20 human breast milk samples are provided from the Milk bank at the neonatal department at Sahlgrenska Hospital in Gothenburg, Sweden. The samples are analyzed for histidine content using the method of Chen et al (Analytica Chimica Acta, Volume 570, Issue 1, 7 Jun. 2006, Pages 109-115).

The samples are pooled to make 4 samples in total containing around 10, 20, 30 and 40 mg histidine per 100 ml milk.

The 4 pooled milk samples are put in 3 test tubes each in amounts of 20 ml per tube. Test products 1, 2 and 3 are put in the respective test tube at an amount of product 1 and 2 to be 10⁷ CFU of the Bacterial strain per tube, and product 3 is put in an equivalent volume as product 1 and 2. The test tubes are put in an anaerobic chamber and incubated in 37° C. over night.

The samples in the test tubes are analyzed for histamine content using the Histamine ELISA kit (Neogen, Lexington, Ky.) according to the manufacturer's instructions.

### Results

Amounts of histamine in pooled human milk samples (mg/100 ml)

| | 10 mg | 20 mg | 30 mg | 40 mg |
|---|---|---|---|---|
| Test product | histidine | histidine | histidine | histidine |
| 1 | 0 | 0 | 0 | 0 |
| 2 | 0.4 | 0.7 | 1.0 | 1.2 |
| 3 | 0 | 0 | 0 | 0 |

The strain of test product 2, L. reuteri ATCC PTA 4659 is selected.

### Example 2

Topical compositions tested, C, I and J. Three different topical compositions were tested containing different components. All compositions were mixed for a total weight of 15 grams, all containing 2% culture of certain histamine producing Lactobacillus reuteri strains, in this case exemplified by L. reuteri ATCC PTA 4659. All components were from AarhusKarlshamn Sweden AB, except Lanolin (Lanolines Stella S. A., Belgium).

| Topical composition | | |
|---|---|---|
| C | I | J |

| Components | % | g | % | g | % | g |
|---|---|---|---|---|---|---|
| MCT | 40 | 6 | 58 | 8.7 | | |
| Lipex Bassol | 38 | 5.7 | | | 75 | 11.3 |
| Lipex205 | 10 | 1.5 | | | | |
| Akogel | 10 | 1.5 | | | 13 | 1.9 |
| Lanolin | | | 40 | 6 | 10 | 1.5 |
| Culture | 2 | 0.3 | 2 | 0.3 | 2 | 0.3 |
| Total | 100 | 15 | 100 | 15 | 100 | 15 |

### Example 3

A topical composition was tested. The composition was mixed to a total weight of 15 grams, containing 2% culture of certain histamine producing Lactobacillus reuteri strains, in this case exemplified by L. reuteri ATCC PTA 4659. All components were from AarhusKarlshamn Sweden AB.

Topical
composition
K

| Components | % | g |
|---|---|---|
| MCT | 48.5 | 7.275 |
| Sunflower oil | 48.5 | 7.275 |
| Silica (silicon dioxide) | 1 | 0.15 |
| Culture | 2 | 0.3 |
| Total | 100 | 15 |

### Example 4

Pilot Study of the Effect of the Probiotic Lactobacillus reuteri on the Treatment of Lactational Mastitis

In the pilot study, women presenting with symptoms of lactational mastitis are randomized, with the intention of 20 complete cases in each group. The women will be identified through incoming telephone calls to the breastfeeding clinic at Sahlgrenska University Hospital East, Gothenburg, Sweden. All midwives working at the breastfeeding clinic have long experience in taking care of women with breastfeeding problems and at least 7.5 higher education points in breastfeeding. The usual routine is that women with mastitis related symptoms are either counseled to home care and expectancy. These women will have a routine follow-up via telephone the day after by a midwife on the breastfeeding clinic. Those patients who are eligible for antibiotic treatment will be invited to come for a visit at the clinic.

Women eligible for this study are those women who are counseled to home care and expectancy. A research midwife will contact eligible women by phone for further information and invite interested women to a first visit at the clinic (the same day). The women will receive further oral and written information on the study and the midwife evaluates if the mother is eligible for the study. This evaluation will be based on a questionnaire and a physical examination.

Women will then be randomized to probiotic treatment or placebo by an electronic database. The treatment of both groups will continue for 14 days. Women will continue breast-feeding during the treatment period. Women will be followed up by visits and telephone interviews for 28 days and fill in a diary regarding their own and their child's symptoms during the first 14 days.

All study participants will be followed by the breastfeeding clinic. This might include follow-up by phone, visit to the breastfeeding clinic or other departments of the hospital and if regarded necessary antibiotic treatment according to the hospitals routine. Independent of follow-up or treatment, women will remain in the study, once included, except if they wish to withdraw from the study.

### Inclusion Criteria

Women presenting with a history of at least 4 hours fever 38° C. and at least 1 point for erythema and 1 point for breast tension according to the Kvist scale and who are recommended home care and expectancy regarding antibiotic treatment by the midwives at the breast feeding clinic
Age ≥18 years
Capable of giving informed consent
Willing to comply with treatment application
Capable of understanding and complying with study protocol requirements
The baby has undergone examination by a pediatrician and is considered healthy
Exclusive breastfeeding

### Exclusion Criteria

Current breast injury/trauma
Current mammary abscesses or other mammary pathology
History of/current breast cancer
Breast surgery in the past month
New pregnancy
Premature baby, born <37 weeks of gestational age
Baby is under the subject of neonatal care
Autoimmune disease (both mother and child)
Known or suspected allergies to any of the components of the study product (both mother and child)
Participation in another investigational drug study within 30 days prior to treatment start.
Use of any other probiotic (oral or local)
Ongoing antibiotic treatment

### Treatment Groups 1) and 2):

1) This group receives probiotic oil containing 10⁸ CFU/10 drops of Lactobacillus reuteri ATCC PTA 4659 for external topical application 3 times/day on each breast for 14 days.
2) This group receives placebo oil for external topical application (same composition of probiotic oil without Lactobacillus reuteri) 3 times/day on each breast for 14 days.

### Results

The topical administration of probiotics are expected to
relief in symptoms (flu-like symptoms, fever, local inflammation resulting in redness, pain and tension of the breast, fissures in the nipple and/or mammary areola) caused by lactational mastitis.
the recovery of Lactobacillus reuteri ATCC PTA 4659 in the breast milk after 14 days treatment.
lower milk counts of staphylococcus and streptococcus.
decreased need of antibiotic treatment.

### Example 5

Detection of the Gene Encoding Histidine Decarboxylase (Hdc) in Lactobacillus reuteri with PCR

### Method:

### Sample Preparation

Bacteria were grown over night in MRS broth at 37° C. The Bacterial suspensions were centrifuged at 3500 rpm for 5 min and 1 μl of the pellet was suspended in 100 μl of PBS.

### PCR Analysis

### Primers:

hdcA425fde (CGTCAYTATCCWGCTCCWGG) (SEQ ID NO: 1) and hdcA867rde (TCCATRTCAGTATCWGGKGT) (SEQ ID NO: 2); Product size 442 bp; The primers were designed from an alignment of hdc from L. reuteri, L. hilgardii, L. buchneri and L. sakei. DreannTaq Green PCR Mastermix (2× Thermo Scientific, article number K1081) was used for the PCR reactions. PCR reactions according to this:

| Mastermix with primers | Volume per reaction |
|---|---|
| Water (PCR quality) | 10.0 μl |
| Primer, forw. (10 pmol/μl) | 1.0 μl |
| Primer, rev. (10 pmol/μl) | 1.0 μl |
| DreamTaq | 12.5 μl |

24.5 μl of the mastermix was mixed with 0.5 μl Bacterial suspension and the following program was run: 95° C. 10 min//30×(95° C. 30 s, 48° C. 30 s, 72° C. 30 s)//72° C. 5 min//4° C. forever.

### Results

The results revealed that a number of strains of L. reuteri from different host origin were positive for the gene encoding histidine decarboxylase (see Table 1).

TABLE 1
A summary of the results from the PCR analysis showing the species,
strain and host origin of the tested Bacteria.

| Species | Strain | Host origin | Presence of hdc gene (PCR) |
|---|---|---|---|
| L. reuteri | ATCC PTA 6475 | Human | + |
| | DSM 17938 | Human | − |
| | 93a | Human | + |
| | F33 | Cat | + |
| | C30 | Cow | + |
| | D276 | Dog | + |

The present invention is not limited to the embodiments disclosed but may be varied and modified within the scope of the following claims.

1. A biologically pure culture of Lactobacillus reuteri selected from the Lactobacillus reuteri DSM 32229, Lactobacillus reuteri DSM 32230, Lactobacillus reuteri DSM 32231, and Lactobacillus reuteri DSM 32232, in a dried form.

2.-3. (canceled)

4. A method for the treatment of mastitis and/or thrush, comprising administering a composition containing one or more lactic acid bacteria strains, wherein the bacteria strains have an ability to convert histidine, present in a topical body fluid, into histamine.

5. The method of claim 4, wherein the composition comprises a lotion, cream, ointment, oil, salve, liniment, embrocation, rub, gel, petroleum jelly, balm, emollient, unguent, or balsam.

6. The method of claim 4, comprising topical administration to skin or mucosa of a mammal.

7. The method of claim, comprising topical administration to mammary glands.

8. The method of claim 4, wherein the one or more lactic acid bacteria strains comprise a Lactobacillus reuteri strain.

9. The method of claim 4, wherein the one or more lactic acid bacteria strains comprise one or more of Lactobacillus reuteri DSM 32229, Lactobacillus reuteri DSM 32230, Lactobacillus reuteri DSM 32231, and Lactobacillus reuteri DSM 32232.

10. The method of claim 4, wherein the one or more lactic acid bacteria strains comprise one or more of Lactobacillus reuteri ATCC PTA 6475, Lactobacillus reuteri ATCC PTA 4659, Lactobacillus reuteri ATCC PTA 5289 and Lactobacillus reuteri ATCC PTA 5290.

11. A composition for treatment of mastitis and/or thrush comprising one or more lactic acid bacteria strains of claim 9, wherein the composition is a lotion, cream, ointment, oil, salve, liniment, embrocation, rub, gel, petroleum jelly, balm, emollient, unguent, or balsam.

12. A composition for treatment of mastitis and/or thrush, comprising one or more lactic acid bacteria strains of claim 10, wherein the composition is a lotion, cream, ointment, oil, salve, liniment, embrocation, rub, gel, petroleum jelly, balm, emollient, unguent, or balsam.

13.-16. (canceled)

17. The method according to claim 4, wherein the composition is topically administered using an adsorbent or non-adsorbent product.

18. The method according to claim 17, wherein the adsorbent or non-adsorbent product is a pad, a wipe and/or a tissue made of adsorbent or non-adsorbent material, whereby the material is suitable as a carrier for the one or more lactic acid bacteria strains, and wherein the adsorbent or non-adsorbent product is adapted to be placed in contact with the mammary gland of the mammal.

19. The method according to claim 18, wherein the adsorbent or non-adsorbent product is contained or surrounded by cotton wadding or wool.

20. The method according to claim 19, wherein the cotton wadding or wool is adapted to provide heat.

21. A method for selecting a lactic acid bacteria strain for use in treating mastitis and/or thrush, wherein the strain has an ability to convert histidine, present in a topical body fluid, into histamine, the method comprising: (a) preparing a lactic acid bacteria sample; and (b) screening for a lactic acid bacteria strain having an active histidine operon by polymerase chain reaction (PCR); and (c) selecting a strain that has an active histidine operon; and/or comprising (a) incubating a sample containing histidine with one or more lactic acid bacteria strains at about 37° C. for 20 to 30 hours; (b) determining the amount of histamine in the sample; and (c) selecting a strain that has an ability to convert histidine to histamine.

22. The method according to claim 21, wherein the method comprises (a) preparing a lactic acid bacteria sample; and (b) screening for a lactic acid bacteria strain having an active histidine operon by polymerase chain reaction (PCR); and (c) selecting a strain that has an active histidine operon, and wherein the PCR uses primer hdcA425fde having the nucleic acid sequence CGTCAYTATCCWGCTCCWGG and primer hdcA867rde having the nucleic acid sequence TCCATRTCAGTATCWGGKGT.


SEQUENCE LISTING (2 sequences)

SEQ ID NO: 1 - DNA, 20 bp, Lactobacillus reuteri
cgtcaytatccwgctccwgg

SEQ ID NO: 2 - DNA, 20 bp, Lactobacillus reuteri
tccatrtcagtatcwggkgt